(19) United States
(12) Patent Application Publication
(10) Pub. No.: US 2012/0158095 A1
(43) Pub. Date: Jun. 21, 2012

(54) Cochlear Implant with Cochleostomy Ground

(75) Inventor: Claude Jolly, Innsbruck (AT)

(73) Assignee: MED-EL ELEKTROMEDIZINISCHE GERAETE GMBH, Innsbruck (AT)

(21) Appl. No.: 13/326,472

(22) Filed: Dec. 15, 2011

Related U.S. Application Data
(60) Provisional application No. 61/423,714, filed on Dec. 16, 2010.

(51) Int. Cl. A61F 11/04; A61N 1/372; A61N 1/05

(52) U.S. Cl. 607/57; 607/137

(57) ABSTRACT

A cochlear implant electrode arrangement is described. An electrode lead is connected to an implantable stimulator and contains electrode wires for carrying stimulation signals from the implantable stimulator. An electrode array penetrates through a cochleostomy opening into a patient cochlea for immersion in cochlear fluid within the cochlea. The electrode array has electrode contacts for delivering the stimulation signals to adjacent neural tissue. A cochleostomy ground contact at the cochleostomy opening separates the electrode lead and the electrode array and provides a return current path for the stimulation signals. There may also be a stimulator ground contact on an outer surface of the implantable stimulator that provides a second return current path for the stimulation signals.

This application claims priority from U.S. Provisional Patent Application 61/423,714, filed Dec. 16, 2010; which is incorporated herein by reference.

### TECHNICAL FIELD

The present invention relates to a ground electrode arrangement for a cochlear implant system.

### BACKGROUND ART

A normal ear transmits sounds as shown in FIG. 1 through the outer ear 101 to the tympanic membrane 102, which moves the bones of the middle ear 103 that vibrate the oval window opening of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

Hearing is impaired when there are problems in the ability to transduce external sounds into meaningful action potentials along the neural substrate of the cochlea 104. To improve impaired hearing, auditory prostheses have been developed. For example, when the impairment is related to operation of the middle ear 103, a conventional hearing aid may be used to provide acoustic-mechanical stimulation to the auditory system in the form of amplified sound. Or when the impairment is associated with the cochlea 104, a cochlear implant with an implanted stimulation electrode can electrically stimulate auditory nerve tissue with small currents delivered by multiple electrode contacts distributed along the electrode.

FIG. 1 also shows some components of a typical cochlear implant system which includes an external microphone that provides an audio signal input to an external signal processor 111 where various signal processing schemes can be implemented. The processed signal is then converted into a digital data format, such as a sequence of data frames, for transmission into an implanted stimulator 108. Besides receiving the processed audio information, the implanted stimulator 108 may also perform additional signal processing such as error correction, pulse formation, etc., and produces stimulation signals (based on the extracted audio information) that is sent through an electrode lead 109 to an implanted electrode array 112. Typically, this electrode array 112 includes multiple electrode contacts 110 on its surface that deliver the stimulation signals to adjacent neural tissue of the cochlea 104 which the brain of the patient interprets as sound. The individual electrode contacts 110 may be activated sequentially, or simultaneously in one or more groups of electrode contacts 110.

The electrical stimulation signals are usually in a monopolar mode between one active electrode contact 110 within the cochlea 104 and a distant extra-cochlear ground contact for the return current. For example, the ground contact may be on the extra-cochlear electrode lead 109 itself, or on a separate branch member. The surface area of the ground contact is usually much larger than that of an individual electrode contact 110 in order to minimize charge density and distribute the return current over a larger area.

Sometimes in some cochlear implant patients, in addition to the desired auditory stimulation, one or more electrode contacts 110 may also create unwanted stimulation of the facial nerve. In some extreme cases, the stimulation to the facial nerve may occur on all the electrode contacts 110. Facial nerve stimulation may occur when the patient fit programming to make the stimulation signals loud enough for optimal auditory perception exceeds the threshold for facial nerve stimulation. One possible reason for this may be because the bone of the cochlear may be sclerotic as in otosclerosis. Other possible reasons could include one or more bone thickness abnormalities of the bone that surrounds the cochlear fluids, or that the facial nerve may have an aberrant course around the exterior of the cochlear bone.

To date the problem has been solved by turning off individual electrode contacts 110 that undesirably stimulate the facial nerve. Another way that the problem has been approached is by using perimodiolar electrode contacts 110 that approach the modiolus and the nerve elements. When the electrode array 112 is displaced toward the inner wall of the cochlea 104, the electrode contacts 110 are presumably at an adequate distance from the facial nerve.

### SUMMARY

Embodiments of the present invention are directed to a cochlear implant electrode arrangement. An electrode lead is connected to an implantable stimulator and contains electrode wires for carrying stimulation signals from the implantable stimulator. An electrode array penetrates through a cochleostomy opening into a patient cochlea for immersion in cochlear fluid within the cochlea. The electrode array has electrode contacts for delivering the stimulation signals to adjacent neural tissue. A cochleostomy ground contact at the cochleostomy opening separates the electrode lead and the electrode array and provides a return current path for the stimulation signals.

In some embodiments, there may also be a stimulator ground contact on an outer surface of the implantable stimulator that provides a second return current path for the stimulation signals. The cochleostomy ground contact and the stimulator ground contact may be switchably selectable, for example, so that the cochleostomy ground contact provides the return current path for electrode contacts which affect the facial nerve, and the stimulator ground contact provides the return current path for all other electrode contacts.

In specific embodiments, the cochleostomy ground contact may span a length of the electrode from outside the cochleostomy opening to inside the cochleostomy opening. The cochleostomy ground contact may be larger than any of the electrode contacts.

Embodiments of the present invention also include a cochlear implant system having an electrode arrangement according to any of the above.

### BRIEF DESCRIPTION OF THE DRAWINGS

FIG. 1 shows a section view of the anatomy of a human ear containing a typical cochlear implant (CI) system.

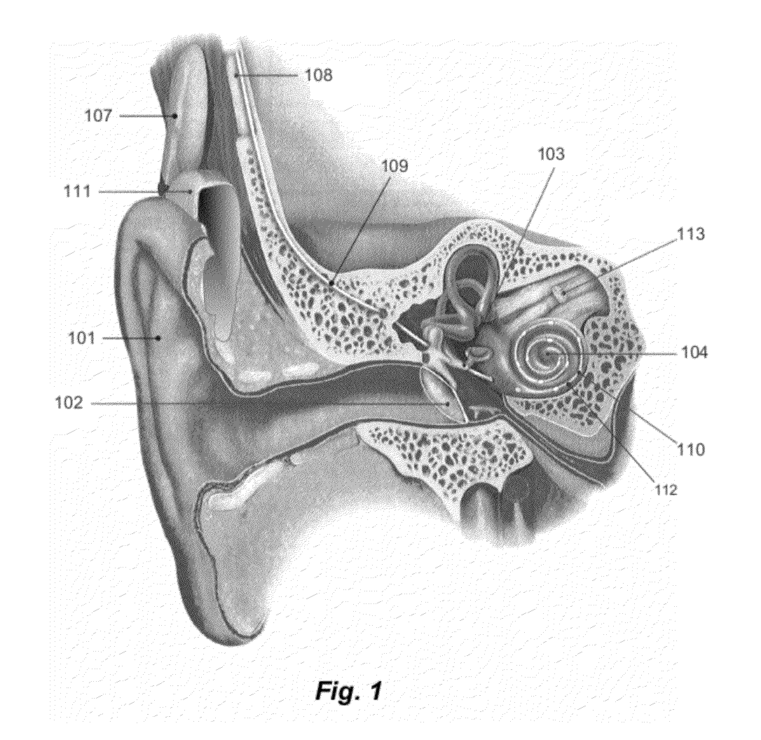

FIG. 2 shows a CI system according to one embodiment of the present invention having a ground electrode at the cochleostomy window.

FIG. 3 shows a CI system according to another embodiment of the present invention having one ground electrode at the cochleostomy window and another ground electrode on the stimulator housing.

### DETAILED DESCRIPTION

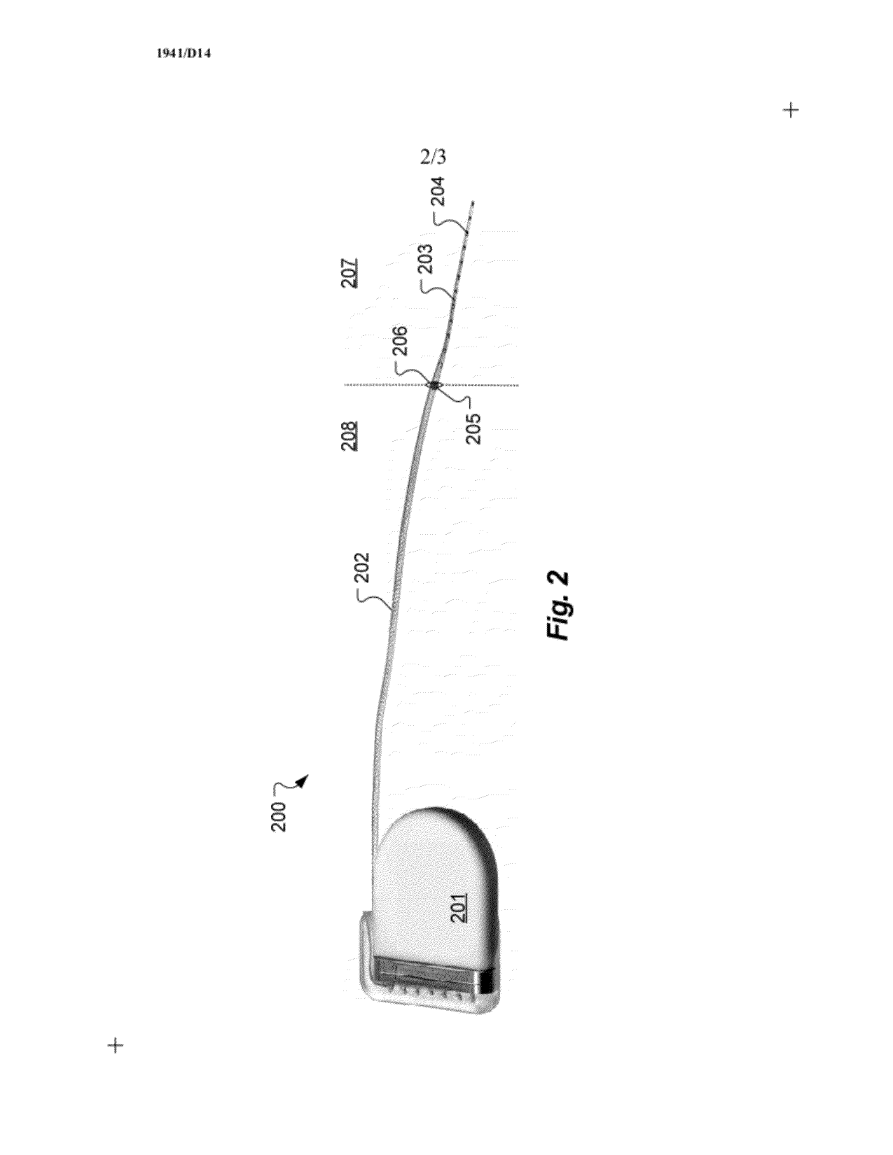

Various embodiments of the present invention are directed to an electrode ground arrangement for cochlear implant systems that avoids facial nerve stimulation. FIG. 2 shows a cochlear implant system 200 according to one embodiment of the present invention. An electrode lead 202 is connected to an implantable stimulator 201 and contains electrode wires for carrying stimulation signals from the implantable stimulator 201. An electrode array 203 penetrates through a cochleostomy opening 206 into a patient cochlea 207 for immersion in cochlear fluid within the cochlea 207. The electrode array 203 has electrode contacts 204 for delivering the stimulation signals to adjacent neural tissue. A cochleostomy ground contact 205 at the cochleostomy opening 206 separates the electrode lead 202 and the electrode array 203 and provides a return current path for the stimulation signals.

The cochleostomy ground contact 205 is much larger than the electrode contacts 204 and spans a length from the middle ear 208 outside the cochleostomy opening 206 to within the cochlea 207 inside the cochleostomy opening 206. This situates the cochleostomy ground contact 205 close to or partially within the scala tympani fluid of the cochlea 207, but still at some relative distance (e.g., 3-4 mm) from the first active electrode contact 204 at the base of the electrode array 203. The return current return path is also more direct from the active electrode contact 204 to the cochleostomy ground contact 205 and has a tendency to remain confined within the conductive scala tympani fluid without escaping through the abnormal surrounding bone connected to the facial nerve.

If we consider bipolar stimulation as referring to a signal applied between two adjacent electrode contacts on an equidistant array of contacts, then the stimulation mode in embodiments of the present invention is not bipolar, but rather quasi monopolar between the cochleostomy ground contact 205 and the first active electrode contact 204 at the base of the electrode array 203, and then for the second active electrode contact 204 the stimulation mode is quasi monopolar +1, (one space between the first electrode contact and the second), then quasi monopolar +2, etc. At some distance from the cochleostomy ground contact 205 the electric field lines from the stimulation pulses stay the same.

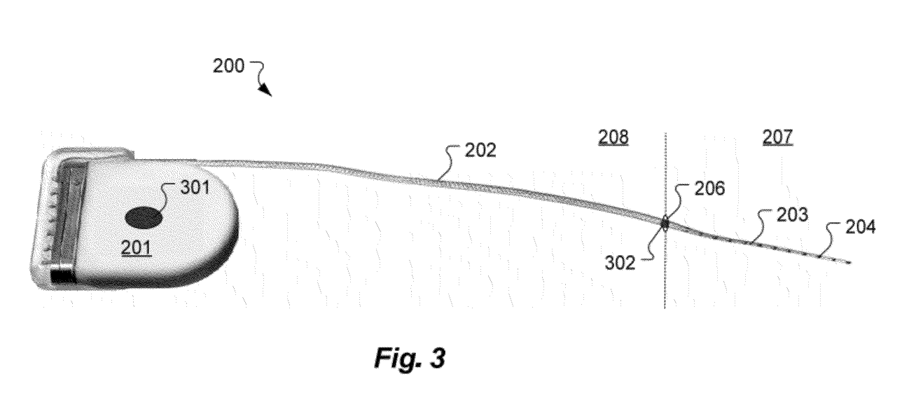

FIG. 3 shows an alternative cochlear implant system 200 according to another embodiment of the present invention having one cochleostomy ground contact 302 at the cochleostomy opening 206 and another stimulation ground contact 301 on the outer surface of the housing of the implantable stimulator 201 that provides a second return current path for the stimulation signals. The cochleostomy ground contact 302 and the stimulator ground contact 301 may be switchably selectable, for example, by system software so that the cochleostomy ground contact 302 provides the return current path for electrode contacts 204 which affect the facial nerve, and the stimulator ground contact 301 provides the return current path for all other electrode contacts 204.

Thus with embodiments of the present invention, all the electrode contacts 204 can be used even if there is some initial stimulation of the facial nerve after the implanted system is turned on. For such adverse reactions, the implicated electrode contacts 204 can be paired with the cochleostomy ground contact 205 or 302 and there will be no unused regions without stimulation along the length of the electrode array 204.

Although various exemplary embodiments of the invention have been disclosed, it should be apparent to those skilled in the art that various changes and modifications can be made which will achieve some of the advantages of the invention without departing from the true scope of the invention.

What is claimed is:

1. A cochlear implant electrode arrangement comprising:
an electrode lead connected to an implantable stimulator and containing a plurality of electrode wires for carrying stimulation signals from the implantable stimulator and;
an electrode array penetrating through a cochleostomy opening into a patient cochlea for immersion in cochlear fluid within the cochlea, the electrode array having a plurality of electrode contacts for delivering the stimulation signals to adjacent neural tissue; and
a cochleostomy ground contact at the cochleostomy opening separating the electrode lead and the electrode array and providing a return current path for the stimulation signals.

2. An electrode arrangement according to claim 1, further comprising:
a stimulator ground contact on an outer surface of the implantable stimulator and providing a second return current path for the stimulation signals.

3. An electrode arrangement according to claim 2, wherein the cochleostomy ground contact and the stimulator ground contact are switchably selectable.

4. An electrode arrangement according to claim 2, wherein the cochleostomy ground contact provides the return current path for electrode contacts which affect the facial nerve, and the stimulator ground contact provides the return current path for all other electrode contacts.

5. An electrode arrangement according to claim 1, wherein the cochleostomy ground contact spans a length of the electrode from outside the cochleostomy opening to inside the cochleostomy opening.

6. An electrode arrangement according to claim 1, wherein the cochleostomy ground contact is larger than any of the electrode contacts.

7. A cochlear implant system having an electrode arrangement according to any of claims 1-5.
